(19) United States
(12) Patent Application Publication
(10) Pub. No.: US 2018/0179482 A1
(43) Pub. Date: Jun. 28, 2018

(54) Flexible Fermentation Vessel and Uses Thereof

(71) Applicant: Brewjacket, Inc., Boulder, CO (US)

(72) Inventors: Aaron Walls, Boulder, CO (US); Evgeniy Tkachenko, Broomfield, CO (US); Matthew Goff, Broomfield, CO (US)

(73) Assignee: Brewjacket, Inc., Boulder, CO (US)

(21) Appl. No.: 15/853,256

(22) Filed: Dec. 22, 2017

Related U.S. Application Data
(60) Provisional application No. 62/438,490, filed on Dec. 23, 2016.

(51) Int. Cl. C12M 1/00; C12C 13/10; C12G 1/036

(52) U.S. Cl. CPC C12M 23/26; C12M 23/20; C12G 1/0206; C12C 13/10

(57) ABSTRACT

The present invention relates to a flexible fermentation, storage, transportation, and dispensing vessel and, more particularly, to a vessel that is structured and/or configured to ferment and/or store a liquid that can be cooled and/or heated and/or pressurized and/or protected from environments that would affect fermentation as a standalone container and/or in conjunction with additional device(s) using a flexible material and/or ports and/or valves.

### RELATED APPLICATION DATA

The present application claims priority to and the benefit of U.S. provisional patent application No. 62/438,490, filed Dec. 23, 2016, and is hereby incorporated by reference in its entirety.

### BACKGROUND OF THE INVENTION

### 1. Field of the Invention

The present invention relates to a flexible fermentation, storage, transportation, and dispensing vessel, and more particularly, to a vessel that is structured and/or configured to ferment and/or store a liquid that can be cooled, heated, pressurized and/or protected from environments that would affect fermentation as a standalone container and/or in conjunction with additional device(s) using a flexible material and/or ports.

### 2. Description of the Related Art

Conventional fermentation and/or storage and/or transportation vessels, such as a carboy, a keg, or a bottle, are rigid containers that can be sealed to contain a liquid as it ferments into alcohol, pressurized to carbonate the liquid, and then store, transport, and serve said liquid after the fermentation is complete. The average brewer can have two to three (or more) fermenters or other containers that they have to use every time they brew, which take up space. The brewer must also clean and sanitize all of the fermenters and transfer the beverage between one or more of them. Each additional manual cleaning, sanitation, and transfer step introduces a typically high risk of introducing infection, other contamination, and/or an undesirable amount of oxygen and/or UV light to the fermentation process. Many of the currently available fermentation containers can also be easily damaged, which happens often. For example, glass or PET carboys typically get dropped and dented.

### SUMMARY OF THE INVENTION

Various embodiments of the present invention may be advantageous in that they may solve or reduce one or more of the potential problems and/or disadvantages discussed above.

Various embodiments of the present invention may exhibit one or more of the following objects, features and/or advantages:

It is a principal purpose and advantage of the present invention to provide a flexible vessel for the fermentation of a liquid. A flexible vessel for fermentation and/or storage and/or transportation of a liquid includes one or more of the following advantages over conventional fermentation vessels (as compared to conventional fermentation vessels): takes up less storage space, is able to be easily disposed after use, can be manipulated into particular shapes to enhance the fermentation process, can be easily sanitized or sterilized, can be easily transported, is difficult to destroy, is difficult to temporarily or permanently damage, or break into multiple pieces by dropping and/or hitting, only requires to be stored in locations that can contain the volume of the liquid (unlike solid fermentation vessels that need to be stored in locations that can accommodate the vessel's shape).

It is another object of the present invention to provide a flexible vessel for the fermentation, storage and/or transportation of the liquid either as a standalone container and/or in conjunction with additional device(s) which perform similar functionalities such as automated fermentation, storage, and/or transportation of the liquid.

In accordance with the foregoing objects and advantages, an embodiment of the present invention is directed at a flexible fermentation vessel including one or more of the following: a flexible material that is used to contain the liquid, one or more ports to interact with the liquid in the vessel and/or between the port and the valve, one or more valves to contain the liquid in the vessel.

The details of one or more embodiments are described below and in the accompanying drawings. Other objects and advantages of the present invention will in part be obvious, and in part appear hereinafter.

### BRIEF DESCRIPTION OF THE DRAWINGS

The present invention will be more fully understood and appreciated by reading the following Detailed Description in conjunction with the accompanying drawings.

FIG. 1 is a schematic representation of a flexible fermentation vessel according to an embodiment.

FIG. 2 is a schematic close up representation of one of the ports/spouts of the flexible fermentation vessel shown in FIG. 1.

### DETAILED DESCRIPTION

The present invention will be more fully understood and appreciated by reading the following Detailed Description in conjunction with the accompanying drawings, wherein like reference numerals refer to like components.

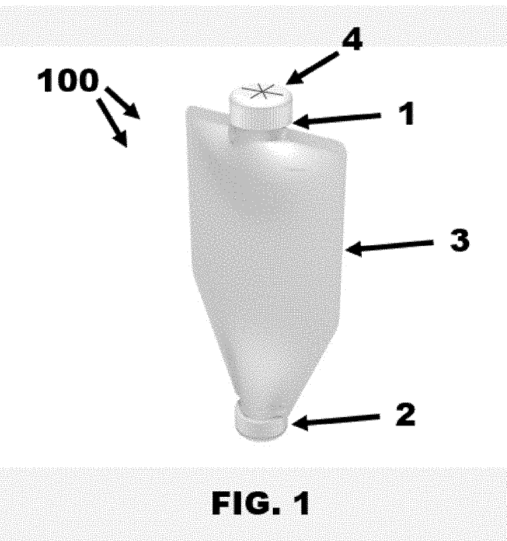

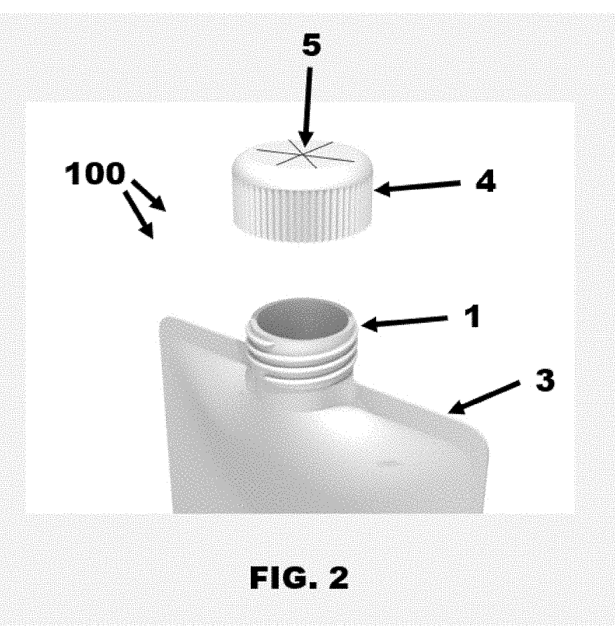

According to an embodiment, a flexible fermentation vessel 100 is shown in FIG. 1 and a close up representation of one of the ports/spouts of the flexible fermentation vessel 100 is shown in FIG. 2. The flexible fermentation vessel 100 can include, but is not limited to, at least two spouts/ports 1, 2, a flexible vessel/bag 3, and at least two valves 4, 5. In one embodiment of attaching the valve 4 to the port/spout 1 of the flexible fermentation vessel 100, the valve 4 is threaded to the spout 1, as shown in FIG. 1 and FIG. 2. The valve 4 may also be attached to the spout 1 by other means, such as snapping/clipping it on (as should be understood by a person of skill in the art in conjunction with a review of this disclosure). At least one valve can include any of the following valve technologies/configurations (as should be understood by those of skill in the art in conjunction with a review of this disclosure): valves that close automatically via an elastic membrane, air, hydraulics, pneumatics, springs, shape memory alloys, bimetals, levers (both electronically controlled and manually controlled) that turn an actuator such as a ball, gates that rise or lower, and/or check valves that release at pressures defined by computer programs or by static physical properties. In one embodiment of a valve technology/configuration, FIG. 2 shows valve 4 with an elastic membrane 5 which closes automatically, and opens when a mating component is inserted into it.

According to an embodiment, the flexible fermentation vessel 100 can be constructed by using one or more materials, exclusively or in combination, such as but not limited to plastics, metals, alloys, ceramics, and composites including, but not limited to, polyethylene and its variants (HDPE, LDPE, polyethylene terephthalate, biaxially-oriented polyethylene terephthalate, among others), polypropylene, nylon, polycarbonate, polystyrene, polyimide films, acrylonitrile butadiene styrene, stainless steel, aluminum, nickel, zinc, chromoly, copper, titanium, silver, tin, platinum, iron, gold, alumina, zirconia, steatite, mullite, cordierite and lava.

According to an embodiment, the flexible fermentation vessel 100 is flexible, disposable, and can repeatedly withstand temperatures as low as 32° F. and as high as 175° F. for sanitation, for example, by hot water inside the flexible fermentation vessel 100. The flexible fermentation vessel 100 can also be impenetrable by or has a high impermeability to oxygen (e.g., <5 cm³-mil/(100 in²-24 HRS-ATM) at 25° C.), carbon dioxide (e.g., <5 cm³-mil/(100 in²-24 HRS-ATM) at 25° C.), water vapor (e.g., <0.5 g-mil/(100 in²-24 HRS) at 37.8° F. and 90% RH) and/or UV light, and can be pressurized. The flexible fermentation vessel 100 can have one or more ports and valves while still offering enough protection against dropping/hitting/damaging the container (as compared with conventional devices). These features can be essential to the fermentation of beverages and are not present in any other conventional food containers or fermenters.

Sanitation is an important factor because it is particularly burdensome for home and commercial fermentation. Most conventional fermenters are not able to withstand temperatures higher than 150° F., and the conventional fermenters must be sanitized with a chemical prior to brewing. Brewers must then clean the fermenter after brewing. However, because the flexible vessel of an embodiment can be disposable, the cleaning step can be eliminated and a sanitation step can be done before the user receives the vessel.

In addition, as the flexible fermentation vessel 100 of an embodiment can be impenetrable by or has a high impermeability to the elements described above, it can also be able to be used as a storage container for a fermented beverage and/or ingredients, such as liquid or dry malt extract, solid grains and additives, malted grain and malted gain extract, yeast, and/or hops, and other ingredients to be transformed into (or can be used to form) a fermented liquid or beverage. As discussed in the Background section above, the average brewer has two to three (or more) fermenters or other containers that they have to use every time they brew, which take up space. The brewer must also clean and sanitize all of the fermenters and transfer the beverage between each of them. Each additional manual cleaning, sanitation, and transfer step has a high risk of introducing infection, other contamination, and an undesirable amount of oxygen and/or UV light to the fermentation process. Many of the currently available fermentation containers are also easily damaged, which happens often. For example, glass or PET carboys get dropped and dented. However, the flexible, disposable vessel of an embodiment can replace two to three (or more) containers with just one unit (vessel) and can solves all of the above listed issues of cleaning, sanitation, and storage.

In one embodiment of a method for manufacturing the vessel is made from one or more layers of high-density polyethylene (HDPE) or low-density polyethylene (LDPE), with an exterior layer or coating made of a mylar, nylon, aluminum, or ethylene vinyl alcohol copolymer (EVOH). HDPE allows the vessel to be flexible and prevents oxygen and carbon dioxide to leave and enter the bag, while mylar protects the bag against UV light. In place of HDPE, LDPE may be used to improve the flexibility and puncture resistance of the bag, while nylon or ethylene vinyl alcohol (EVOH) may be used to mitigate oxygen and carbon dioxide penetration.

What is claimed is:

1. A flexible fermentation vessel system comprising:
a flexible vessel comprising an exterior surface, and an interior surface defining an interior space containing a fermented liquid or ingredients that can be mixed and used to form a fermented liquid;
wherein the flexible vessel is formed from one or more of the following components: polyethylene, polypropylene, nylon, ethylene vinyl alcohol, polycarbonate, polystyrene, polyimide films, and acrylonitrile butadiene styrene.

2. The flexible fermentation vessel of claim 1, wherein the flexible vessel is formed from a polyethylene component.

3. The flexible fermentation vessel of claim 2, wherein the polyethylene component comprises high-density polyethylene, low-density polyethylene, polyethylene terephthalate, or biaxially-oriented polyethylene terephthalate.

4. The flexible fermentation vessel of claim 1, wherein the exterior surface comprises a coating formed from a mylar, nylon, aluminum, or ethylene vinyl alcohol copolymer.

5. The flexible fermentation vessel of claim 1, further comprising at least one port configured to allow access to the interior space.

6. The flexible fermentation vessel of claim 5, further comprising a valve connected to the at least on port.

7. The flexible fermentation vessel of claim 6, wherein the valve comprises an elastic membrane.

8. The flexible fermentation vessel of claim 1, wherein the flexible vessel is structured to have an impermeability to oxygen as follows: <5 cm³-mil/(100 in²-24 HRS-ATM) at 25° C.

9. The flexible fermentation vessel of claim 1, wherein the flexible vessel is structured to have an impermeability to carbon dioxide as follows: <5 cm³-mil/(100 in²-24 HRS-ATM) at 25° C.

10. The flexible fermentation vessel of claim 1, wherein the flexible vessel is structured to have an impermeability to water vapor as follows: <0.5 g-mil/(100 in²-24 HRS) at 37.8° F. and 90% RH.

11. A method of using a flexible fermentation vessel comprising the steps of:
providing a flexible fermentation vessel comprising an exterior surface, and an interior surface defining an interior space, wherein the flexible vessel is formed from one or more of the following components: polyethylene, polypropylene, nylon, ethylene vinyl alcohol, polycarbonate, polystyrene, polyimide films, and acrylonitrile butadiene styrene;
placing a fermented liquid or ingredients that can be mixed and used to form a fermented liquid in the interior space.

12. The method of claim 11, further comprising the step of mixing the fermented liquid or fermented liquid ingredients.

13. The method of claim 12, wherein the step of mixing further comprising the step of manipulating the flexible fermentation vessel into a plurality of different shapes.

14. The method of claim 11, further comprising the step of increasing the pressure of the fermented liquid or fermented liquid ingredients within the interior space to a pressure above sea level ambient pressure.

15. The method of claim 11, further comprising the step of increasing the temperature of at least a portion of the interior space up to 100 degrees Celsius.

16. The method of claim 11, further comprising the step of decreasing the temperature of at least a portion of the interior space down to between 0 degrees Celsius and 114.1 degrees Celsius.

17. The method of claim 11, wherein the flexible vessel is formed from a polyethylene component.

18. The method of claim 11, wherein the exterior surface comprises a coating formed from a mylar, nylon, aluminum, or ethylene vinyl alcohol copolymer.

19. The method of claim 11, wherein the flexible vessel further comprises at least one port configured to allow access to the interior space.

20. The flexible fermentation vessel of claim 5, wherein the flexible vessel further comprises a valve connected to the at least on port.
